(19) United States
(12) Patent Application Publication
(10) Pub. No.: US 2021/0319328 A1
(43) Pub. Date: Oct. 14, 2021

(54) AUTOMATIC QUERY CONSTRUCTION FOR KNOWLEDGE DISCOVERY

(71) Applicant: BENEVOLENTAI TECHNOLOGY LIMITED, London (GB)

(72) Inventors: Daniel Paul Smith, London (GB); Jiajie Zhang, Oxfordshire (GB)

(73) Assignee: BENEVOLENTAI TECHNOLOGY LIMITED, London (GB)

(21) Appl. No.: 17/270,359

(22) PCT Filed: Jun. 17, 2019
(86) PCT No.: PCT/GB2019/051673

§ 371 Date: Feb. 22, 2021

(30) Foreign Application Priority Data
Aug. 23, 2018 (GB) 1813742.2

(51) Int. Cl. G06N 5/02; G06F 16/903

(52) U.S. Cl. CPC G06N 5/02; G06F 16/90335

(57) ABSTRACT

A system for discovering biological knowledge patterns of interest is described. The system comprises: a receive module configured to receive information defining a base pattern and a generalised base pattern, the base pattern comprising one or more entity nodes each representing a biological entity and one or more biological relationships indicated between the nodes, the generalised base pattern being related to the base pattern by virtue of replacing at least one entity node representing a respective biological entity by an associated set node representing a set of biological entities that includes the respective biological entity; a query module configured to generate a first query portion that, in combination with the generalised base pattern, defines a first query that retrieves a first set of results including the base pattern; and a control module configured to cause the query module to generate a second query portion that, in combination with the first query, defines a second query that retrieves a second set of results including the base pattern.

### CROSS-REFERENCE TO RELATED PATENT APPLICATIONS

This patent application is the 35 U.S.C. 371 national stage of International Patent Application PCT/GB2019/051673 filed 17 Jun. 2019; which claims the benefit of priority to GB 1813742.2 filed 23 Aug. 2018, which is incorporated by reference herein for all purposes.

The present application relates to a system and computer-implemented method for automatically constructing database queries to help support a user in discovering interesting sets of related entities. The approach is particularly well suited to assist a drug discovery scientist in finding biological knowledge patterns of interest.

### BACKGROUND

Knowing which questions to ask is often half the challenge in knowledge discovery activities. It can therefore be a significant barrier to knowledge discovery that users have little to go on when directing their search, and consequently there can be a combination of a lack of guidance and information overload. This creates inefficiencies in the knowledge discovery process, with knowledge discoverers having to manually come up with search queries based on their own knowledge, recent findings or literature review, or a hunch. Patterns and undiscovered connections remain hidden in the vast amount of information that is currently searchable, and the rate at which new discoveries can be made is restricted.

An approach for constructing queries automatically is needed so that the process of knowledge discovery can be enhanced and made more efficient.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter.

The present disclosure provides systems and methods for discovering knowledge patterns, and biological entities, and sets of biological entities of interest, for example for use in drug discovery.

In a first aspect, the present disclosure provides a system for discovering biological knowledge patterns of interest. The system comprises: a receive module configured to receive information defining a base pattern and a generalised base pattern, the base pattern comprising one or more entity nodes each representing a biological entity and one or more biological relationships indicated between the nodes, the generalised base pattern being related to the base pattern by virtue of replacing at least one entity node representing a respective biological entity by an associated set node representing a set of biological entities that includes the respective biological entity; a query module configured to generate a first query portion that, in combination with the generalised base pattern, defines a first query that retrieves a first set of results including the base pattern; and a control module configured to cause the query module to generate a second query portion that, in combination with the first query, defines a second query that retrieves a second set of results including the base pattern.

Some embodiments of the system have additional features. In one or more embodiments, the control module is configured to cause the query module to generate the second query portion only if the first set of results comprises a number of results that is outside a target range. In one or more embodiments, the system comprises a generalise module configured to generate the generalised base pattern by: receiving the base pattern; receiving an instruction to replace the at least one entity node of the base graph by the associated set node; and replacing the at least one entity node of the base pattern by the associated set node. In this case, at least one of the base pattern and the instruction may be based on a user input. In one or more embodiments, each query portion comprises a further set node representing a set of biological entities and a relationship between the further set node and one of the entity nodes or set nodes of the generalised base pattern. In one or more embodiments, the query module is configured to generate a query portion by searching a relationship schema database storing sets of biological entities and ways in which they can be related. In one or more embodiments, the control module is configured to remove a query portion if it prevented retrieval of the base pattern. In one or more embodiments, the control module is configured to cause the query module to generate further query portions that still retrieve the base pattern until an output pattern is reached that retrieves a number of results within the target range. In this case, the control module may be configured to output the output pattern or its results or both. In one or more embodiments, the system is configured to maximise a reward R of the output pattern. In this case, the system may be configured to maximise the reward R of the output pattern by selecting the output pattern from a plurality of output patterns based on their respective rewards R. In one or more embodiments, the system comprises a function approximator such as a neural network trained to maximise the reward R. In this case, the function approximator may comprise one or more neural networks comprising reinforcement learning algorithms. In one or more embodiments, the reward R of the output pattern comprises a combination of rewards r of each query portion that lead to the output pattern. In one or more embodiments, the query module is configured to maximise a reward, r, each time it generates a query portion. In a second aspect, the present disclosure provides a computer-implemented method for discovering biological knowledge patterns of interest. The method comprises receiving information defining a base pattern and a generalised base pattern, the base pattern comprising one or more entity nodes each representing a biological entity and one or more biological relationships indicated between the nodes, the generalised base pattern being related to the base pattern by virtue of replacing at least one entity node representing a respective biological entity by an associated set node representing a set of biological entities that includes the respective biological entity; generating a first query portion that, in combination with the generalised base pattern, defines a first query that retrieves a first set of results including the base pattern; and causing the query module to generate a second query portion that, in combination with the first query, defines a second query that retrieves a second set of results including the base pattern.

Some embodiments of the method have additional features. In one or more embodiments, the method comprises causing the query module to generate the second query portion in response to the first set of results comprising a number of results that is outside a target range. In one or more embodiments, the method comprises comprising generating the generalised base pattern by: receiving the base pattern; receiving an instruction to replace the at least one entity node of the base graph by the associated set node; and replacing the at least one entity node of the base pattern by the associated set node. In this case, at least one of the base pattern and the instruction may be based on a user input. In one or more embodiments, each query portion comprises a further set node representing a set of biological entities and a relationship between the further set node and one of the entity nodes or set nodes of the generalised base pattern. In one or more embodiments, the method comprises generating a query portion by searching a relationship schema database storing sets of biological entities and ways in which they can be related. In one or more embodiments, the method comprises removing a query portion if it prevented retrieval of the base pattern. In one or more embodiments, the method comprises causing the query module to generate further query portions that still retrieve the base pattern until an output pattern is reached that retrieves a number of results within the target range. In one or more embodiments, the method comprises outputting the output pattern or its results or both. In one or more embodiments, the method comprises maximising a reward R of the output pattern.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1 is a schematic diagram illustrating a base pattern comprising a combination of biological entities and relationships between them;

FIG. 2 is a schematic diagram illustrating a first query pattern comprising the above base pattern and a first query portion;

FIG. 3 is a schematic diagram illustrating a second query pattern comprising the first query pattern and a second query portion;

FIG. 4 is a is a schematic diagram illustrating a third query pattern comprising the second query pattern and a third query portion;

FIG. 5 is a flow chart illustrating a worked example of generating a constrained query pattern for retrieving a number of results within a target range including the base pattern;

FIG. 6 is a flow chart illustrating a computer-implemented method of generating a constrained query pattern for retrieving a number of results within a target range including the base pattern;

FIG. 7 is a schematic diagram illustrating a module view of a system for performing the above computer-implemented method; and

FIG. 8 is a schematic diagram of hardware suitable for implementing a system according to the present disclosure.

Common reference numerals are used throughout the figures to indicate similar features.

### DETAILED DESCRIPTION

Embodiments of the present invention are described below by way of example only. These examples represent the best ways of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

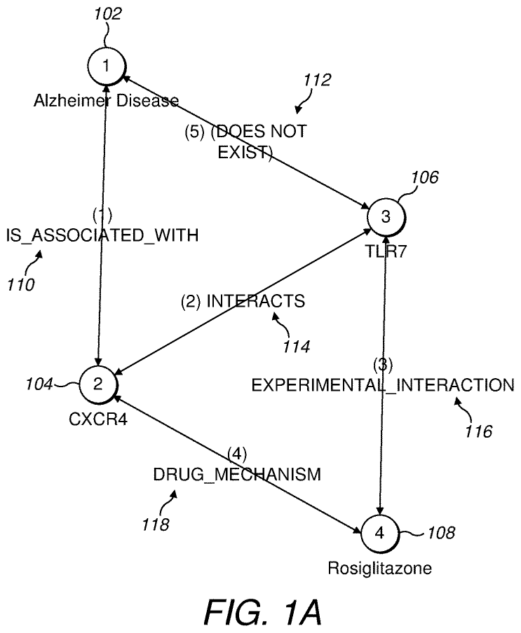

FIG. 1A shows a simple knowledge pattern 100 comprising four biological entities and ways in which they are related. The biological entities are: a disease, two genes, and a drug. In this example, the disease is Alzheimer's disease represented by a first entity node 102, the genes are CXCR4 and TLR7, represented by a second entity node 104 and a third entity node 106, and the drug is rosiglitazone represented by a fourth entity node 108. An entity node is a node that represents an entity such as Alzheimer's disease or the gene CXCR4. The four biological entities and the ways they are related are selected to represent a interesting pattern, in this case a known treatment mechanism for Alzheimer's disease. In general, a biological knowledge pattern is a knowledge pattern comprising biological entities, such as genes, diseases, proteins, families of genes, diseases or proteins, and so on, and connections between them showing how they are related, for example relationships of being associated with each other, being involved in a common biological pathway or a common disease treatment mechanism, or relationships in the sense of being known not to be associated with each other. More generally, a knowledge pattern may be defined as a set of entity nodes each representing an entity connected together by relationships.

Relationships between the biological entities are included in the knowledge pattern according to a relationship schema which defines categories or types of entities and the possible relationships between them. For example, according to an example pattern schema, a disease may be associated with a gene, or a gene may have an interaction with another gene. For example, the knowledge pattern of FIG. 1A includes the relationship that Alzheimer's disease is associated with the gene CXCR, and indicates this using a text indication ‘IS_ASSOCIATED_WITH’ 110 and a double ended arrow. By contrast, there is no association between Alzheimer's disease and the gene TLR7, so the relationship is regarded as an absence of an association between the entities, and is indicated using a text indication ‘DOES NOT EXIST’ 112 and a double ended arrow.

Similarly, the two genes CXCR4 and TLR7 are related by virtue of having an interaction with each other. This is included in the knowledge pattern of FIG. 1A using a text indication ‘INTERACTS’ 114 and a double ended arrow. The gene TLR7 and the drug rosiglitazone are related by having an experimental interaction, so this relationship is included in the knowledge pattern and indicated using a text indication ‘EXPERIMENTAL_INTERACTION’ 116 and a double ended arrow. Finally, the gene CXCR4 and the drug rosiglitazone are related by virtue of the gene being a known primary target of the drug, and this relationship is included in the knowledge pattern, indicated using a text indication ‘DRUG_MECHANISM’ 118 and a double ended arrow.

The knowledge pattern illustrates known relationships between a small set of specific biological entities. This known pattern may be referred to as a base pattern and can be used to form the basis for constructing a search query for discovering potentially interesting knowledge patterns. In this context, a base pattern is simply a biological knowledge pattern that is used as a starting point for generating a search query as described below. The base pattern is generally small: for example it may comprise four biological entities.

Referring to FIG. 1B, the base pattern of FIG. 1A is generalised so that at least one of the biological entities is replaced with a set or category of biological entities to which the at least one biological entity belongs. In the example of FIG. 1B, the gene CXCR4 is replaced with the set of genes 120. Similarly, the gene TLR7 is replaced by the set of genes 122 and the drug rosiglitazone is replaced by the set of drugs 124. The resulting pattern may be referred to as a generalised base pattern because one or more of the biological entities of the base pattern have been generalised by replacing them with a set of biological entities to which they belong. Since the nodes 120, 122 and 124 represent sets of entities, they may be referred to as set nodes. It is noted that in other examples, a generalised base pattern may additionally or alternatively be generalised by virtue of generalising one or more of the relationships between the biological entities.

At this stage, the generalised base pattern defines a query because it may be used to search for combinations of biological entities that fall within its scope. As such, it may be referred to as a search query. For example, in the case of FIG. 1B, the search query would be looking for a combination of two genes and a drug that fits the requirements of the generalised base graph, including all the relationships between the entities.

If a query were executed on the basis of the generalised base pattern of FIG. 1B, an unmanageably large number of results, such as hundreds of thousands of results, would be returned. In order to generate a useful query, the generalised base pattern is constrained to reduce the number of results.

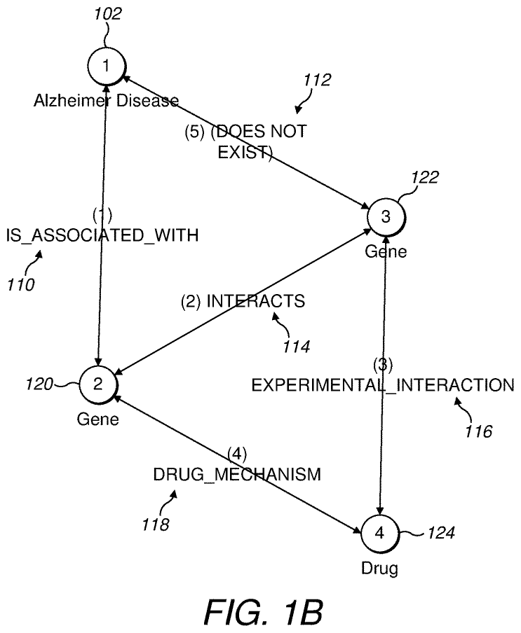

Referring to FIG. 2, a query pattern 200 comprises the generalised base pattern together with a query portion 202 that constrains the search. The query portion 202 comprises a category of biological processes represented by set node 204, the category of biological processes having an association 206 with the gene CXCR4 and an association 208 with the gene TLR7. The query pattern 200 can be used to define a search query looking for a combination of two genes, a drug, and a biological process that fits the requirements of the query graph 200, including all the relationships between the entities. Any patterns falling within the scope of the query pattern 200 will show up as results of this query. It will be appreciated that the results of this query would be a subset of any results of a search performed on the basis of the generalised base pattern of FIG. 1B because of the constraint provided by the query portion 202.

In an example, when a query defined by the query pattern 200 is executed, the number of results is 172,000. This number of results is still unmanageably large for a user to review so there is a problem of information overload. The search needs to be further constrained in order to reduce the number of results towards a more manageable target range.

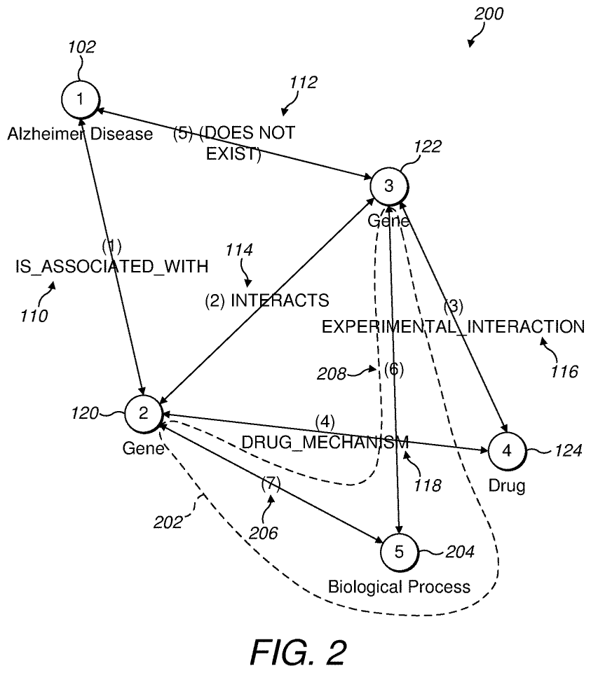

Referring to FIG. 3, a query pattern 300 with a further query portion 302 is shown. This further constrains the query by providing an additional constraint that must be satisfied. The query portion 302 comprises a category of biological pathways represented by a set node 304, the category of biological pathways having an association 306 with the gene TLR7 and not having an association with the gene CXCR4. The absence of the association with the gene CXCR4 is indicated by a text indication ‘DOES NOT EXIST’ 308. The query pattern 300 can be used to define a query looking for a combination of two genes, a drug, a biological process, and a pathway that fits the requirements of the query graph 300, including all the relationships between the entities. Any patterns falling within the scope of the query pattern 300 will show up as results of this query, and the results are a subset of the results from the search defined by query pattern 200. In the present example, a query executed on the basis of query pattern 300 retrieves 23,120 results. Again, this number is unmanageable and should be reduced

To be within a target range by adding another constraint to the search. A target range of 10-250 results would be suitable. In examples, a target range may be specified by the user. For example, depending on the task at hand, the user may want to specify a range of 10 to 20 results or 1000 or more results.

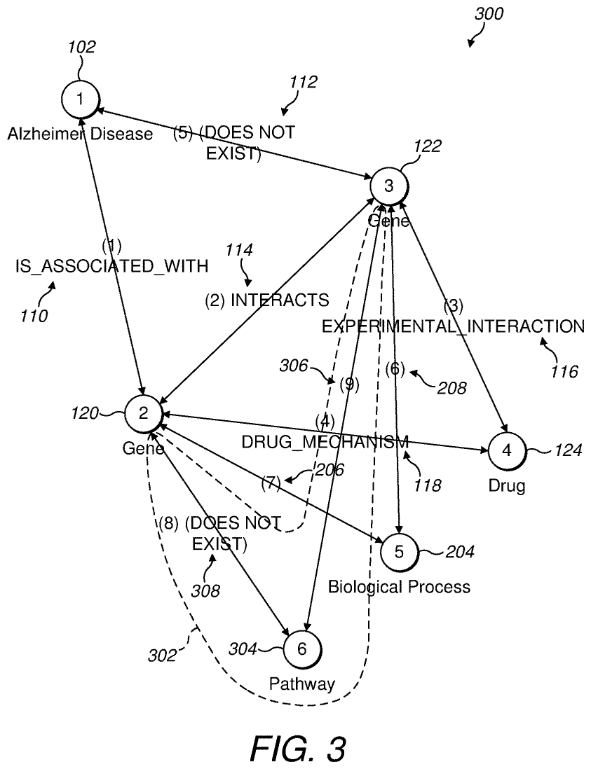

Referring to FIG. 4, a query pattern 400 with another query portion 402 is shown. The query portion 402 comprises a category of a first protein family represented by a set node 404, the first protein family being a protein family to which the gene TLR7 belongs, and a second protein family represented by a set node 406, the second protein family being a protein family to which the gene CXCR4 belongs. The query pattern 400 can be used to define a query looking for a combination of two genes, a drug, a biological process, a pathway, and two protein families that fits the requirements of the query graph 400, including all the relationships between the entities. Any patterns falling within the scope of the query pattern 400 will show up as results of this query, and the results are a subset of the results from the search defined by query pattern 300. In the present example, a query executed on the basis of query pattern 400 retrieves 213 results. Using a target range of 10-250 results, 213 results is within the target range and the search can be stopped. The final pattern (i.e. the query pattern 400) and its 213 results can be treated as the output of this process.

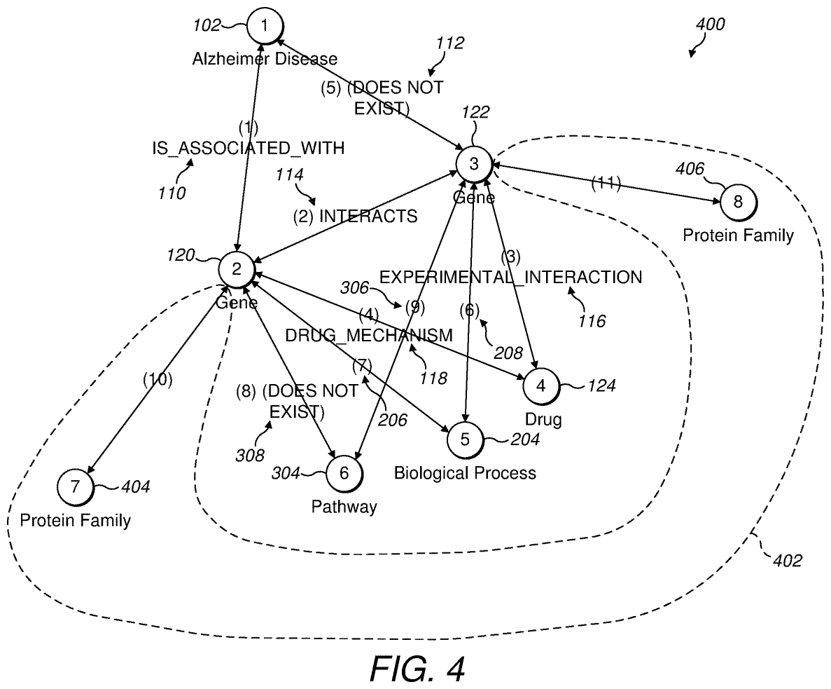

A worked example of this process is summarised in a computer-implemented method 500 shown in the flow chart of FIG. 5. To start, a generalised base pattern such as the generalised base pattern of FIG. 1A is received at step 502. A first query portion is generated at step 504 that, in combination with the generalised base pattern, defines a first query that is executed at step 506. Optionally, a first query pattern comprising the generalised base pattern and the first query portion may be generated, but this is not essential. At step 508 it is determined that the number of results is outside a target range. The process therefore continues by constraining the search by generating a second query portion at step 510. In combination with the first query pattern, the second query portion defines a second query which is executed at step 512. A second query pattern comprising the generalised base pattern, the first query portion and the second query portion may optionally be generated. It is determined at step 514 that the number of results is still outside the target range. The search is constrained again by generating a third query portion at step 516 to define a third query which is executed at step 518. Finally, it is determined at step 520 that the number of results is now within the target range and the process ends.

Although not shown in the flow chart of FIG. 5, each time a new query portion is generated, it is also checked whether the results of the associated query include the base pattern. If they do not, then the most recent query portion is removed and a different one is tried. To enable this, information defining not only the generalised base pattern but also the base patter are received as part of the method 500. The goal of the process is to generate a query pattern that, when executed as a query, retrieves a number of results within a target range and still includes the base pattern. It is important to retrieve the base pattern because the aim of the search is to find similar patterns to the base pattern. If the base pattern appears among the results, this indicates that the new query pattern is capable of finding results similar to the base pattern. If the base pattern is not found in the results, this indicates that the new query pattern is excessively constrained or constrained in a way that is not helpful for finding patterns similar to the base pattern.

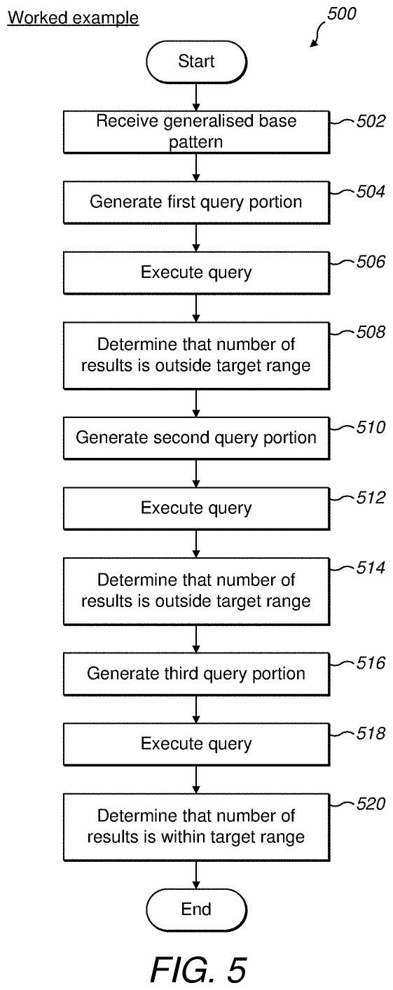

With the worked example of FIG. 5 in mind, a general computer-implemented method 600 will now be described with reference to the flow chart of FIG. 6. To start, a generalised base pattern is received at step 602, and at step 604 a query portion is generated. In combination with the generalised base pattern, the query portion defines a search query that is executed at step 606. The results of this query are subjected to two decision steps. It is determined at a first decision step 608 whether the results include the base pattern. If they do not, then the recent query portion is removed at step 610. If the results do include the base pattern, then it is determined at a second decision step 612 whether the number of results is within a target range. If it is, then the process ends and a manageable number of results that includes the base pattern has been reached. If the number of results is not within the target range, then the process cycles back to step 604 and another query portion is generated. The process continues until a sufficiently constrained query is reached that returns a number of results within the target range, including the base pattern. The sufficiently constrained query that returns a number of results within the target range, including the base pattern, may be referred to as the output query. If a query pattern corresponding to the output query is generated, this pattern may be referred to as the output pattern.

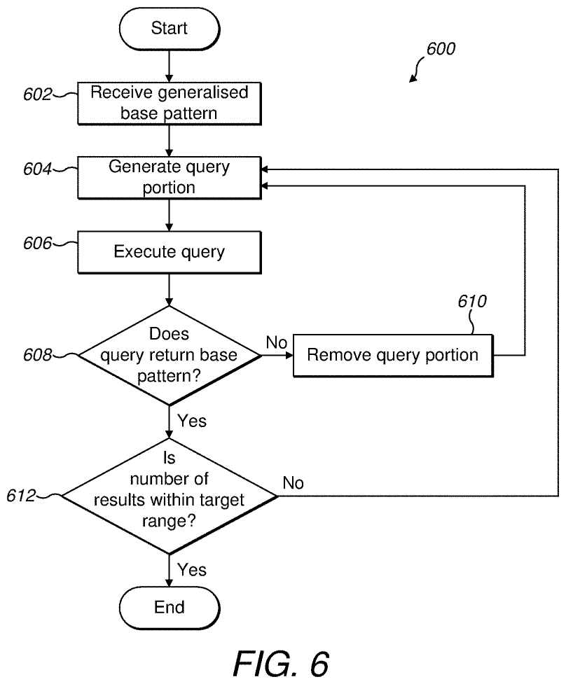

This process may be performed by the system 700 shown in FIG. 7. The system 700 comprises a receive module 702 configured to receive information defining a base pattern and a generalised base pattern, the base pattern comprising one or more entity nodes each representing a biological entity and one or more biological relationships indicated between the nodes, the generalised base pattern being related to the base pattern by virtue of replacing at least one entity node representing a respective biological entity by an associated set node representing a set of biological entities that includes the respective biological entity; a query module 704 configured to generate a first query portion that, in combination with the generalised base pattern, defines a first query that retrieves a first set of results including the base pattern; and a control module 706 configured to cause the query module to generate a second query portion that, in combination with the first query, defines a second query that retrieves a second set of results including the base pattern. The query module 704 may be connected to a relationship schema database 708 storing a relationship schema defining categories or types of entities and the possible relationships between them. In this case, the query module 704 may refer to the relationship schema database 708 in order to ensure that query portions respect the relationship schema. The query module 704 may also be connected to a pattern database 710 which stores known biological knowledge patterns. In this case, the query module may be configured to search the pattern database for results when executing a query.

For each query Q that is executed, a reward r may be defined. In general, the reward r may be a function F of the query Q and the number n of results it retrieves. For example, a reward r₁ of a first query Q₁ may be defined as r₁=F(Q₁,n₁). In general, we may say that a reward for an i^{th} query is:

rᵢ=F(Qᵢ,nᵢ)

A total reward R may then be defined for a series of queries from a first query Q₁ that retrieves a very large number of results to an Nₜₕ query that retrieves a number n_{N} of results that is within a target range and includes the base pattern. The total reward R comprises a combination of the individual rewards rᵢ and may be defined as:

$$R = {\sum }_{i = 1}^{N} {r}_{i}$$

In examples according to the present disclosure, a query pattern is generated whilst maximising the total reward R. This may be achieved computationally, for example by performing a Monte Carlo random search and selecting a query pattern with a highest total reward. In this case, available computing power for the computations is configured to accommodate exponential growth of the search space with the number of possible query portions.

Alternatively, the query that maximizes the total reward R may be found by converting the problem of determining an output query pattern (i.e. the problem of determining a query pattern that defines a query returning a number of results within a target range, including the base pattern) into a Markov Decision Process, and finding the optimal policy of the Markov Decision Process using standard reinforcement learning algorithms. The optimal query can then be found by following the optimal policy.

We define the Markov Decision Process (MDP) as following:
State set: all possible database queries and associated query results given a fixed pattern database and a relationship schema database. The starting state is always the query Q₀ associated with the generalised base pattern and its results. The terminating states are those that either do not contain the base pattern or those that have a number of results below a predefined number (e.g. within a target range).
Action set: all allowed query portions that, in combination with an existing query pattern, define a new query.
State transition probability given an action: implicitly defined by the pattern database. The state transition probability of state a and b (included in the state set) is when observing state a, the probability of transition into state b by executing a query. The state transition is thus, the state changes after executing a query.
Reward of a state transition given an action: defined by the reward function F.
Discount factor: a real value number between 0 and 1, indicating the difference in importance of future and immediate rewards.

As the state transition probabilities are implicitly defined by the knowledge graph database, it is suitable in examples to use one of the so-called model-free control algorithms to find the optimal policy. Due to the large number of states, function approximation may be required to speed up the learning and bypass the memory limitation. Details of the algorithm can be found in Reinforcement Learning: An Introduction second edition (Richard S. Sutton and Andrew G. Barto).

Automatic query pattern generation may be used to find new patterns of entities and their relationships that are similar to the base pattern. In this way, the technique of the present disclosure may be used to infer previously unknown relationships between entity pairs. It could also reveal new and alternative relationships among the entities of the base pattern, thus providing further evidence and biologically plausible explanations for the inferred relationships.

The rewards may be defined to reward certain desired characteristics of the queries and/or the number of results they return. For example, it may be desired to reward queries that are associated with query patterns having two genes with a common biological process. This may increase the likelihood that the genes belong to the same biological process behind the gene. In another example, it may additionally or alternatively be suitable to reward a pattern having a first gene being related to a first biological pathway and a second gene being related to a second biological pathway. This is known as targeting multiple mechanisms or pathways in the field of drug discovery. In a yet further example, it may additionally or alternatively be suitable to reward a pattern having multiple genes related to a common tissue. This would increase the likelihood of finding results in which the genes are all associated with processes in the same tissue, thereby increasing the likelihood that the genes of the results belong to a same biological mechanism involved in the disease. The rewards may also be defined to penalise whenever a further query portion or constraint is added to a query pattern in order to discourage overly complex patterns. In an example, every query portion that introduces a specific biological entity receives a penalty of −2.5, and all other query portions receive a penalty of −1.

Examples of the present disclosure remove the bias of a drug discovery scientist when building queries. Since queries are built automatically in the examples, surprising or unfamiliar query patterns can be generated, thereby opening up the possibility of generating new queries and discovering new knowledge patterns. The bias originates from humans' limited understanding of biology and pharmacology which is inherently relied upon when a drug discovery scientist builds queries manually. The construction of queries by a machine also saves time because the queries are built automatically, for example by running a program for a computer.

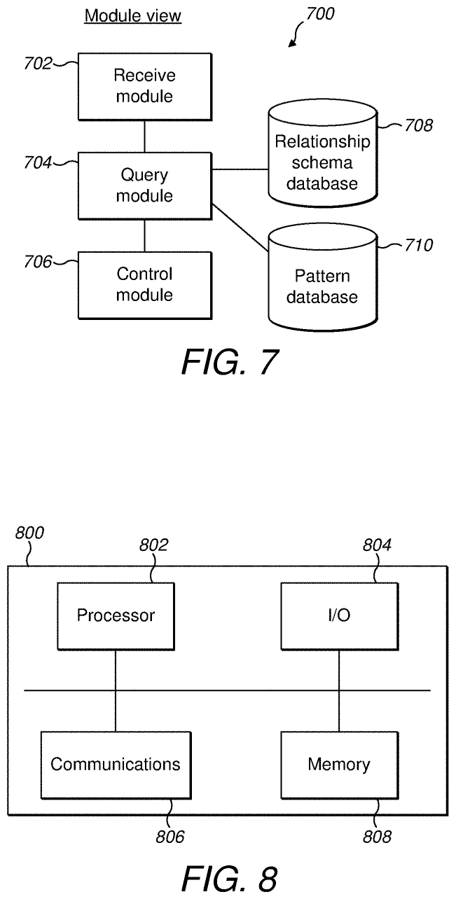

Referring to FIG. 8, the above system 700 may be implemented using hardware 800. The hardware 800 includes a processor 802, an input/output device 804, a communications module 806, and memory 808. The memory 808 may store a program that when executed causes the processor 802 to implement a method comprising receiving information defining a base pattern and a generalised base pattern, the base pattern comprising one or more entity nodes each representing a biological entity and one or more biological relationships indicated between the nodes, the generalised base pattern being related to the base pattern by virtue of replacing at least one entity node representing a respective biological entity by an associated set node representing a set of biological entities that includes the respective biological entity generating a first query portion that, in combination with the generalised base pattern, defines a first query that retrieves a first set of results including the base pattern; and causing the query module to generate a second query portion that, in combination with the first query, defines a second query that retrieves a second set of results including the base pattern.

In the embodiment described above the server may comprise a single server or network of servers. In some examples the functionality of the server may be provided by a network of servers distributed across a geographical area, such as a worldwide distributed network of servers, and a user may be connected to an appropriate one of the network of servers based upon a user location.

The above description discusses embodiments of the invention with reference to a single user for clarity. It will be understood that in practice the system may be shared by a plurality of users, and possibly by a very large number of users simultaneously.

The embodiments described above are fully automatic. In some examples a user or operator of the system may manually instruct some steps of the method to be carried out.

In the described embodiments of the invention the system may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices.

Further, as used herein, the term “exemplary” is intended to mean “serving as an illustration or example of something”.

Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art. What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A system for discovering biological knowledge patterns of interest, the system comprising:
a receive module configured to receive information defining a base pattern and a generalised base pattern, the base pattern comprising one or more entity nodes each representing a biological entity and one or more biological relationships indicated between the nodes, the generalised base pattern being related to the base pattern by virtue of replacing at least one entity node representing a respective biological entity by an associated set node representing a set of biological entities that includes the respective biological entity;
a query module configured to generate a first query portion that, in combination with the generalised base pattern, defines a first query that retrieves a first set of results including the base pattern; and
a control module configured to cause the query module to generate a second query portion that, in combination with the first query, defines a second query that retrieves a second set of results including the base pattern.

2. The system of claim 1, wherein the control module is configured to cause the query module to generate the second query portion only if the first set of results comprises a number of results that is outside a target range.

3. The system of claim 1, comprising a generalise module configured to generate the generalised base pattern by:
receiving the base pattern;
receiving an instruction to replace the at least one entity node of the base graph by the associated set node; and
replacing the at least one entity node of the base pattern by the associated set node.

4. The system of claim 3, wherein at least one of the base pattern and the instruction is based on a user input.

5. The system of claim 1, wherein each query portion comprises a further set node representing a set of biological entities and a relationship between the further set node and one of the entity nodes or set nodes of the generalised base pattern.

6. The system of claim 1, wherein the query module is configured to generate a query portion by searching a relationship schema database storing sets of biological entities and ways in which they can be related.

7. The system of claim 1, wherein the control module is configured to remove a query portion if it prevented retrieval of the base pattern.

8. The system of claim 1, wherein the control module is configured to cause the query module to generate further query portions that still retrieve the base pattern until an output pattern is reached that retrieves a number of results within the target range.

9. The system of claim 8, wherein the control module is configured to output the output pattern or its results or both.

10. The system of claim 8, wherein the system is configured to maximise a reward R of the output pattern.

11. The system of claim 10, wherein the system is configured to maximise the reward R of the output pattern by selecting the output pattern from a plurality of output patterns based on their respective rewards R.

12. The system of claim 11, comprising a function approximator, such as a neural network, trained to maximise the reward R.

13. The system of claim 12, wherein the function approximator comprises one or more neural networks comprising reinforcement learning algorithms.

14. The system of claim 10, wherein the reward R of the output pattern comprises a combination of rewards r of each query portion that lead to the output pattern.

15. The system of claim 1, wherein the query module is configured to maximise a reward, r, each time it generates a query portion.

16. A computer-implemented method for discovering biological knowledge patterns of interest, the method comprising:
receiving information defining a base pattern and a generalised base pattern, the base pattern comprising one or more entity nodes each representing a biological entity and one or more biological relationships indicated between the nodes, the generalised base pattern being related to the base pattern by virtue of replacing at least one entity node representing a respective biological entity by an associated set node representing a set of biological entities that includes the respective biological entity;
generating a first query portion that, in combination with the generalised base pattern, defines a first query that retrieves a first set of results including the base pattern; and
causing the query module to generate a second query portion that, in combination with the first query, defines a second query that retrieves a second set of results including the base pattern.

17. The method of claim 16, comprising causing the query module to generate the second query portion in response to the first set of results comprising a number of results that is outside a target range.

18. The method of claim 16, comprising generating the generalised base pattern by:
receiving the base pattern;
receiving an instruction to replace the at least one entity node of the base graph by the associated set node; and
replacing the at least one entity node of the base pattern by the associated set node.

19. The method of claim 18, wherein at least one of the base pattern and the instruction is based on a user input.

20. The method of claim 16, wherein each query portion comprises a further set node representing a set of biological entities and a relationship between the further set node and one of the entity nodes or set nodes of the generalised base pattern.

21. The method of claim 16, comprising generating a query portion by searching a relationship schema database storing sets of biological entities and ways in which they can be related.

22. The method of claim 16, comprising removing a query portion if it prevented retrieval of the base pattern.

23. The method of claim 16, comprising causing the query module to generate further query portions that still retrieve the base pattern until an output pattern is reached that retrieves a number of results within the target range.

24. The method of claim 23, comprising outputting the output pattern or its results or both.

25. The method of claim 23, comprising maximising a reward R of the output pattern.
